(19) United States
(12) Patent Application Publication
(10) Pub. No.: US 2016/0018242 A1
(43) Pub. Date: Jan. 21, 2016

(54) METHOD AND APPARATUS FOR FITNESS DEVICE

(71) Applicant: Nicholas Webb, Redding, CA (US)

(72) Inventor: Nicholas Webb, Redding, CA (US)

(21) Appl. No.: 14/332,430

(22) Filed: Jul. 16, 2014

(51) Int. Cl. G01C 22/00; A61B 5/11

(52) U.S. Cl. CPC G01C 22/006; A61B 5/1118

(57) ABSTRACT

A ring shaped structure having a ring shaped inner channel, and a ball which is located inside the ring shaped structure and which rolls in the ring shaped channel. The ring shaped structure may be comprised of a first ring shaped structure and a second ring shaped structure which are attached together to form the ring shaped structure. The ring shaped structure may have an inner diameter; and the apparatus may further include a handle which spans the inner diameter of the ring shaped structure. The apparatus may further include a first attachment device; and a second attachment device; wherein the first attachment device connects to the second attachment device inside of the handle to attach the first ring shaped structure to the second ring shaped structure. The apparatus may include a device for measuring movement of the ball. The device for measuring movement of the ball includes an accelerometer.

### FIELD OF THE INVENTION

This invention relates to improved methods and apparatus concerning fitness devices.

### BACKGROUND OF THE INVENTION

Wrist wearable monitoring technologies, devices, or products such as the “Nike+ Fuel Band” (trademarked) and the “FitBit” (trademarked) are known. The “Nike+ FuelBand” (trademarked) is worn on a particular wrist of a person, and has a three axis accelerometer in it. The three axis accelerometer tracks all movements of the particular wrist on which it is located. The “Nike+ Fuelband” (trademarked) measures steps a person takes and also how many calories the person burns based off a profile the person creates with his or her height and weight.

The problem with the “Nike+ Fuelband” (trademarked) and other wearable wrist technologies is that the only data input is coming from the subject human wrist. Correspondingly, the information about a workout is arbitrary and often times erroneous. For example, a vigorous workout may consist of a treadmill or an elliptical machine. Because the hand and wrist of a person are holding onto a stabilizing bar, despite the rigorous workout, the wearable device does not communicate much if any physical movement. Even conventional workouts such as walking or jogging are not consistently measured as some individuals hold connected devices such as iPhones (trademarked) or other smart phones while running or jogging thereby limiting wrist movement.

Samsung Galaxy Gear smartwatch or FitBit (trademarked), a wearable fitness device—is poised to explode over the next few years, according to a recent report from Swedish telecommunications market researcher Berg Insight (trademarked). By 2017, companies will ship more than sixty-four million wearable technology devices. That includes thirty-five million smart watches, a significant bump from the four hundred thousand that are expected to be sold this year.

According to Nitin Bhas, a senior analyst at United Kingdom based Juniper Research (trademarked), 2014 will be “the watershed year for wearables” in terms of roll outs and market traction. “The industry as a whole now acknowledges wearable computing as the next big thing and players ranging from chipset manufacturers to handset vendors are developing products within the wearable segment,” Bhas says. The entry of Google Glass (trademarked) and Samsung's (trademarked) smart watch, along with rumored products coming from Apple (trademarked) and Microsoft (trademarked), validates this segment and indicates the future market opportunity for wearables.

### SUMMARY OF THE INVENTION

One or more embodiments of the present invention provide a handheld exercise apparatus, method, and/or system to work with wearable technologies or devices that monitor a person's body movement.

One or more embodiments of the present invention are especially configured in order to work with wearable technologies so as to provide an accurate and measurable workout that achieves aerobic and kinetic fitness benefits. In order to activate the accelerometer of a wearable device or handheld smartphone, one or more embodiments of the present invention are configured to work in accordance with a range of exercises that are readily measured by an accelerometer and/or other movement sensors. One or more embodiments achieve this by positioning a person's hand on a centrally located handle that demands rotational and thrusting movements that achieve fitness benefits while concurrently providing more accurate and meaningful measurements to the handheld smart phone or wearable technology.

In one or more embodiments, a ball that is captured in a large tubular ring structure encourages the subject or person to use both small and large rotational movements and thrusting movements to create a cadence of ball movement. This ball movement may include a ball rolling in an inner channel of a ring torus or hollow donut structure around an inner perimeter of the ring torus or hollow donut structure. These exercises are then accurately measured and communicated through one or more wearable technologies or devices. This provides a significant benefit to the user by providing them a more accurate measurement and catalog of fitness progression and fitness regimen compliance.

Although an exercise device of one or more embodiments of the present invention does not require any interplay with digital devices to be functional, its movements have been configured to correspond with the way in which an accelerometer measures arm movement.

Another embodiment of the present invention includes the permanent installation of an accelerometer on a body structure of a device or alternatively an accelerometer located in the ball. In this embodiment no wearable device would be required in order to monitor and measure body movement. This would provide a standalone integrated fitness device that includes the fitness apparatus and the accelerometer or other suitable monitoring device.

The benefits of monitoring movement consistently are significant and include the ability for connected devices to aggregate data from a wearable wrist sensor or onboard data from an integrated configuration or design so as to measure the progression of fitness routines in order to determine the accurate level of exercise. In one or more embodiments, this aggregated data of a particular user is stored and compared with other data from other users or historical data. Comparing data with data from other users provides a means to add game mechanics to the apparatus by way of movement competition. Studies have showed that game mechanics can increase utilization and performance of fitness devices as humans are inherently social and competitive. This data is so well known that large organizations are now deploying game mechanics in their health and wellness programs to drive compliance to fitness, weight loss and other health programs.

One or more embodiments of the present application provide a ring shaped structure having a ring shaped inner channel; and a ball which is located inside the ring shaped structure and which rolls in the ring shaped channel. The ring shaped structure may be comprised of a first ring shaped structure and a second ring shaped structure which are attached together to form the ring shaped structure. The ring shaped structure may have an inner diameter; and the apparatus may further include a handle which spans the inner diameter of the ring shaped structure.

The apparatus may further include a first attachment device; and a second attachment device; wherein the first attachment device connects to the second attachment device inside of the handle to attach the first ring shaped structure to the second ring shaped structure. The first attachment device and the second attachment device may be substantially perpendicular to the handle.

The apparatus may include a device for measuring movement of the ball. The device for measuring movement of the ball includes an accelerometer. The device for measuring movement of the ball may include electronic memory for storing data related to the movement of the ball. The device for measuring movement of the ball may include a transmitter for transmitting out data related to the movement of the ball. The device for measuring movement of the ball may include a port for interfacing with a computer device for downloading data related to movement of the ball.

The apparatus may further include a wearable electronic device configured to be attached to a human hand, and which monitors movement of the human hand, when the wearable electronic device is attached to the human hand.

In at least one embodiment, a method is provided which may include gripping a ring shaped structure with a human hand; and moving the human hand while gripping the ring shaped structure; wherein the ring shaped structure has a ring shaped inner channel, and a ball is located in the ring shaped inner channel; and wherein the ball rolls in the ring shaped inner channel when the human hand is moved while gripping the ring shaped structure. The ring shaped structure may have other features previously described.

The method may further include measuring movement of the ball, storing data concerning the movement of the ball in electronic memory, and sending out data concerning movement of the ball. The method may further include attaching a wearable electronic device to the human hand, and monitoring movement of the human hand, when the wearable electronic device is attached to the human hand and while the human is gripping the ring shaped structure.

### BRIEF DESCRIPTION OF THE DRAWINGS

FIG. 1 shows a top perspective view of an apparatus in accordance with an embodiment of the present invention;

FIG. 2 shows a bottom perspective view of the apparatus of FIG. 1;

FIG. 3 shows a top view of the apparatus of FIG. 1;

FIG. 4 shows a bottom view of the apparatus of FIG. 1;

FIG. 5 shows a right side view of the apparatus of FIG. 1;

FIG. 6 shows a left side view of the apparatus of FIG. 1;

FIG. 7 shows a front view of the apparatus of FIG. 1;

FIG. 8 shows a rear view of the apparatus of FIG. 1;

FIG. 9A shows a front view of a structure which is part of the apparatus of FIG. 1;

FIG. 9B shows a rear view of the structure of FIG. 9A;

FIG. 10 shows a front view of the structure of FIG. 9A;

FIG. 11A shows a left side view of the structure of FIG. 9A;

FIG. 11B shows a right side view of the structure of FIG. 9A;

FIG. 12A shows a sectional view of the structure of FIG. 9A;

FIG. 12B shows a closeup view of part of the sectional view of FIG. 12A;

FIG. 13A shows a front view of a further structure which is part of the apparatus of FIG. 1;

FIG. 13B shows a rear view of the further structure of FIG. 13A;

FIG. 14 shows a top view of the further structure of FIG. 13A;

FIG. 15 shows a bottom view of the further structure of FIG. 13A;

FIG. 16A shows a right side view of the further structure of FIG. 13A;

FIG. 16B shows a left side view of the further structure of FIG. 13A;

FIG. 17 shows a right side view of the apparatus 1 taken apart, with the structure of FIG. 9A, the further structure of FIG. 13A, and a ball shown taken apart from each other;

FIG. 18 shows a front view of the apparatus 1 taken apart, with the structure of FIG. 9A, the further structure of FIG. 13A, and the ball of FIG. 17 shown taken apart from each other;

FIG. 19 shows a front perspective view of the apparatus 1 taken apart, with the structure of FIG. 9A, the further structure of FIG. 13A, and the ball of FIG. 17 shown taken apart from each other;

FIG. 20A shows a bottom view of the apparatus 1, with lines for sectional views of FIG. 20A and FIG. 20B;

FIG. 20B shows a sectional view along the line AA shown in FIG. 20A;

FIG. 20C shows a section view along the line BB shown in FIG. 20A;

FIG. 21 shows a top perspective view of another apparatus in accordance with an embodiment of the present invention; and

FIG. 22 shows a block diagram of an apparatus, method, and/or system in accordance with another embodiment of the present invention.

### DETAILED DESCRIPTION OF THE DRAWINGS

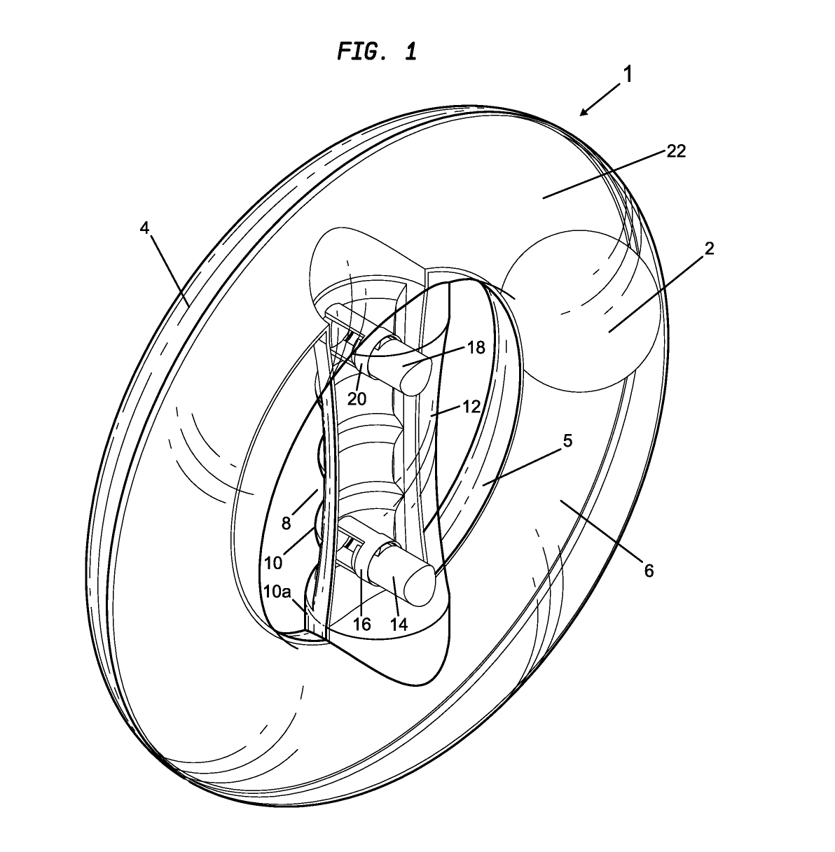

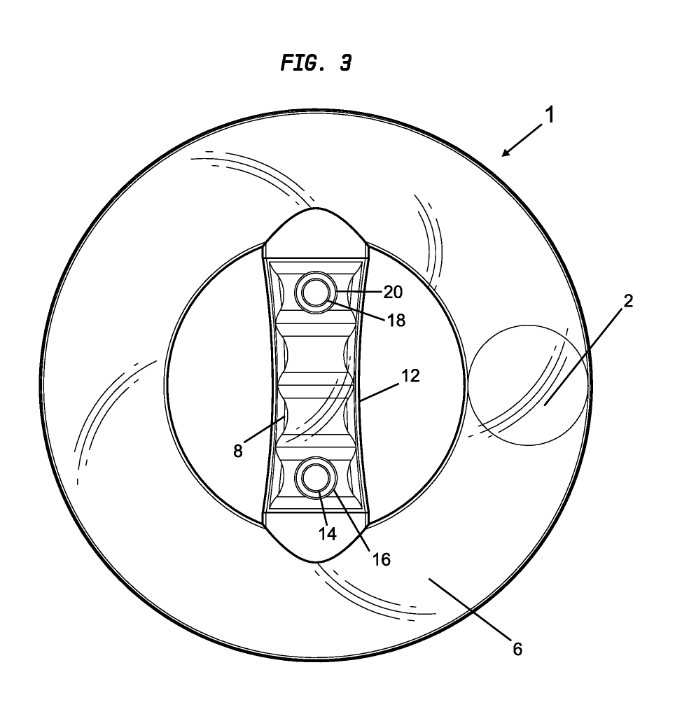

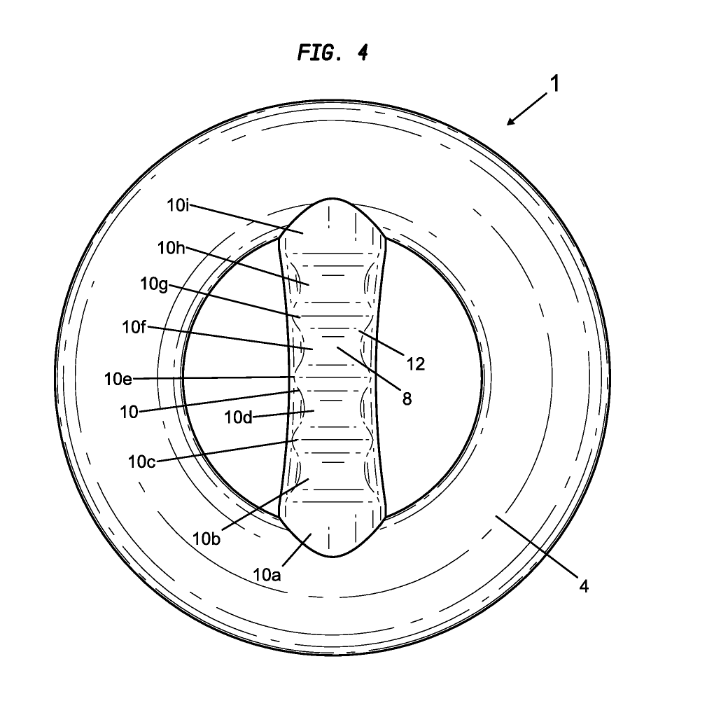

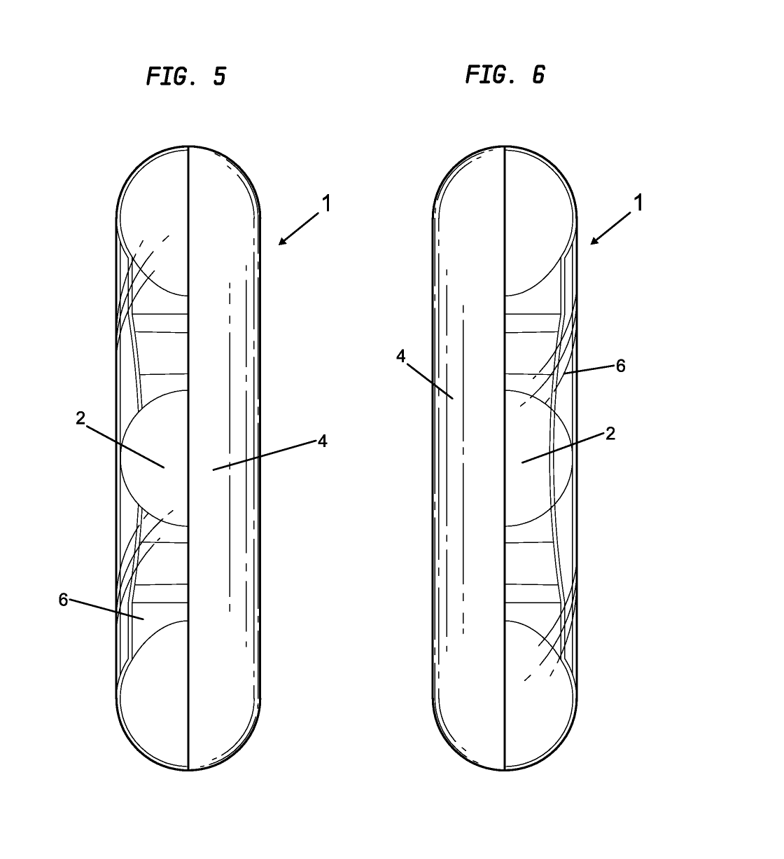

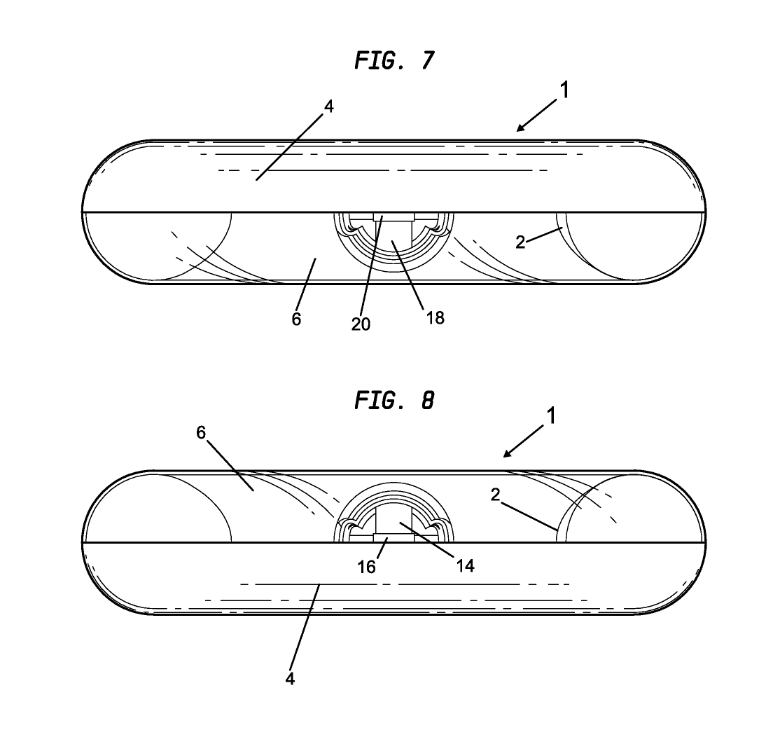

FIGS. 1, 2, 3, 4, 5, 6, 7, and 8, show top perspective, bottom perspective, top, bottom, right side, left side, front, and rear views, respectively, of the apparatus 1.

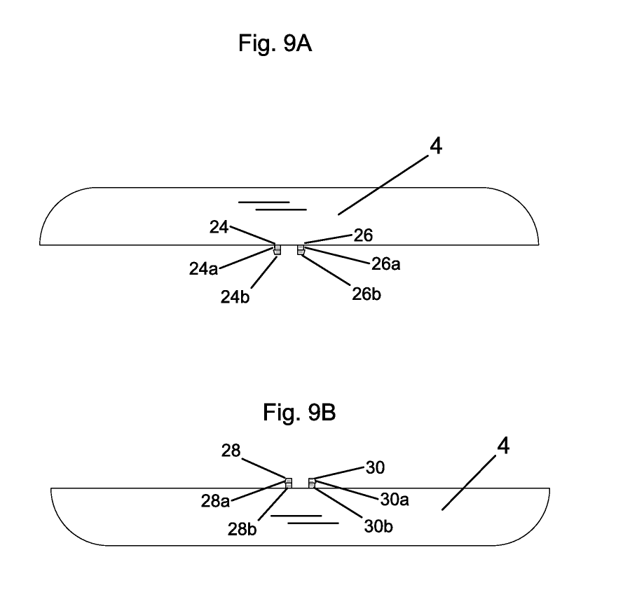

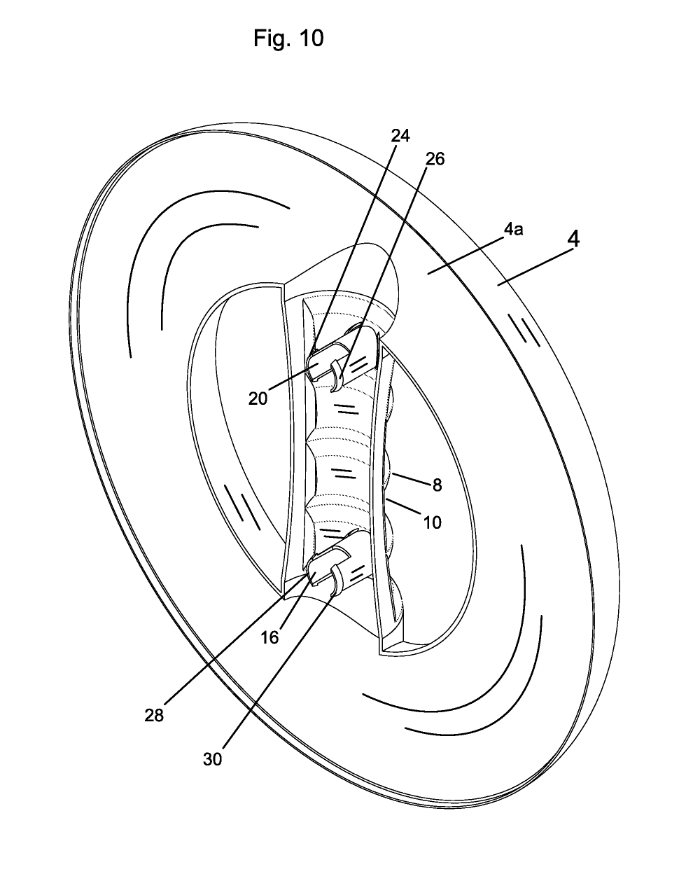

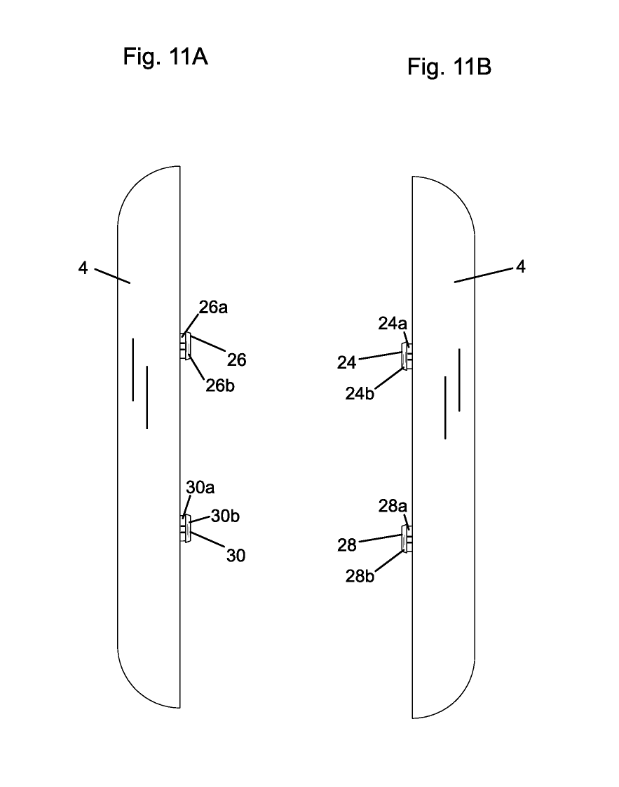

FIGS. 9A-9B, 10, 11A-11B, and 12A-12B show a front view, a rear view, a top perspective view, a left side view, a right side view, a sectional view and a closeup view, respectively, of a structure 4 which is part of the apparatus 1 of FIG. 1.

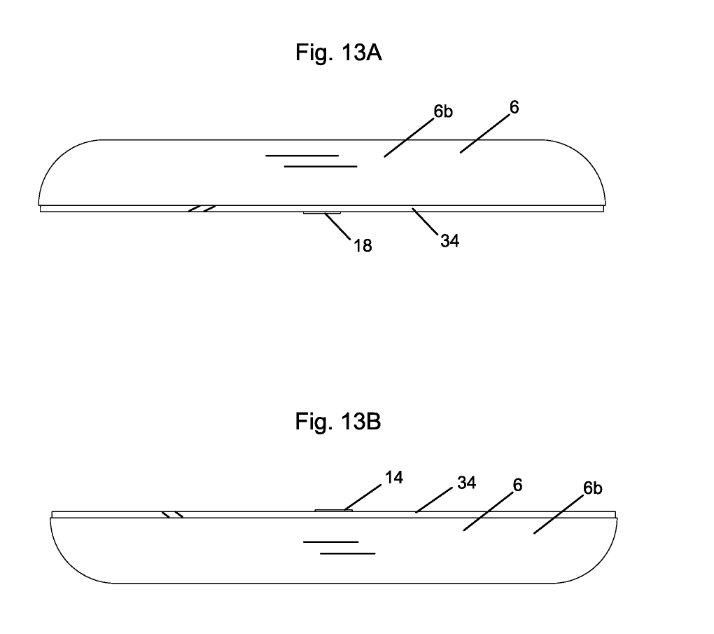

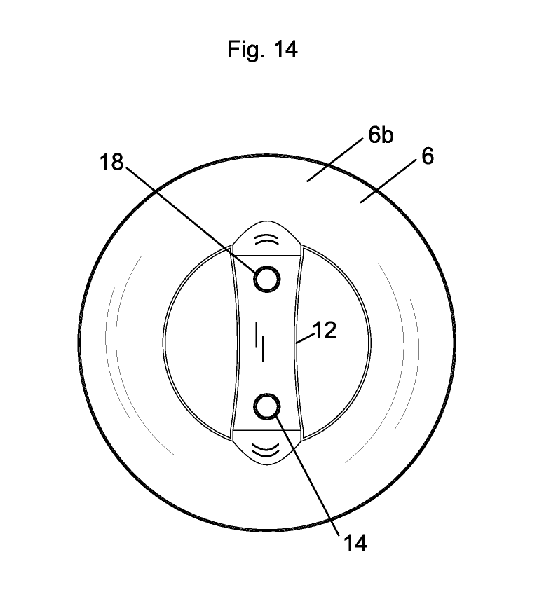

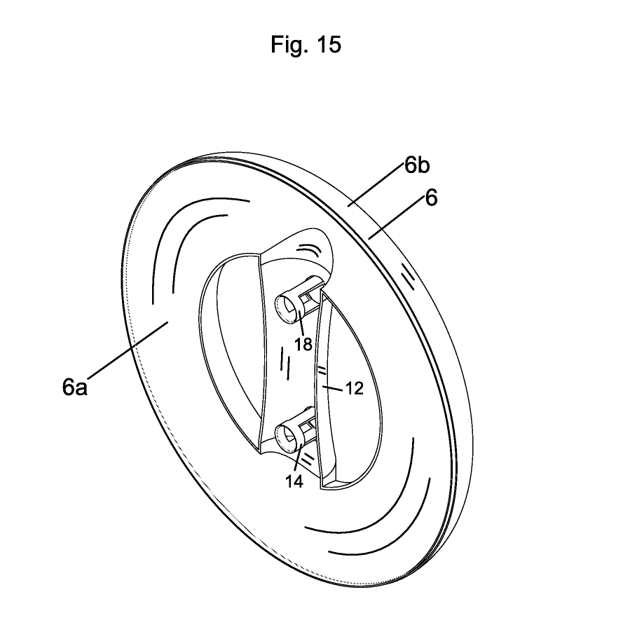

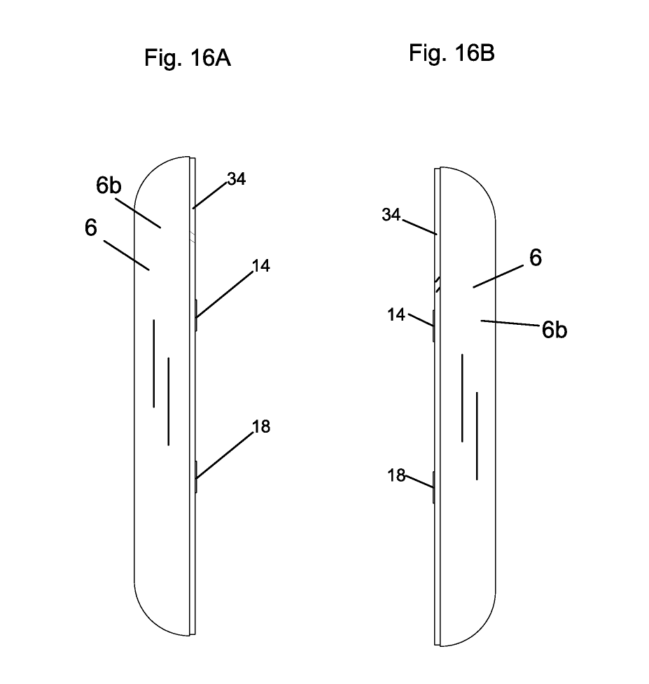

FIGS. 13A-13B, 14, 15, 16A-16B show a front view, a rear view, a top view, a bottom view, a right side view, and a left side view, respectively, of a further structure 6 which is part of the apparatus 1 of FIG. 1.

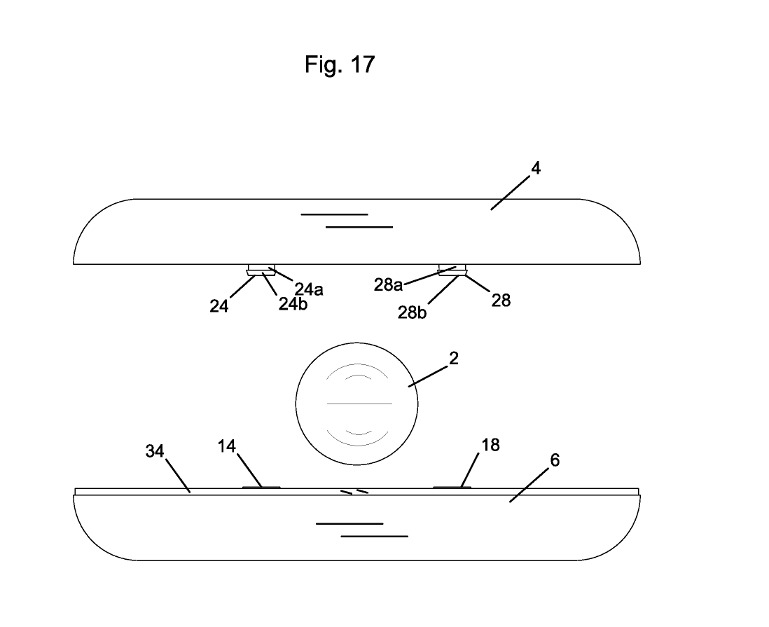

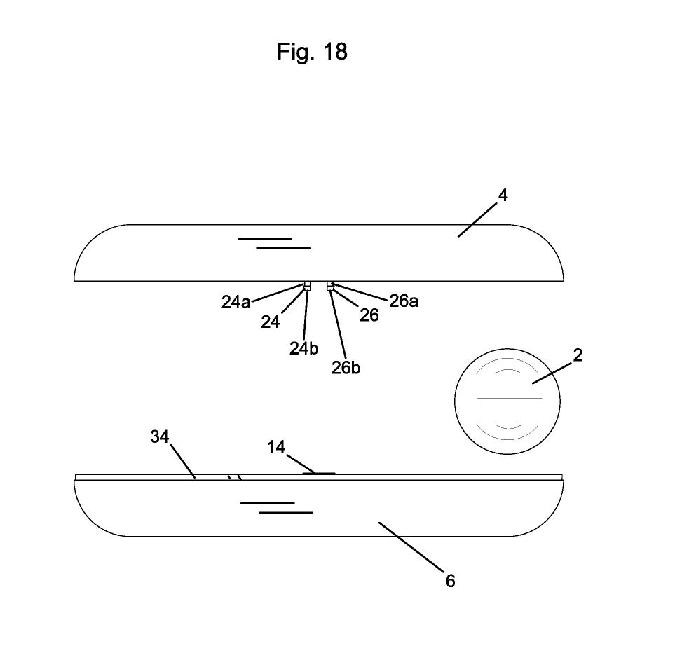

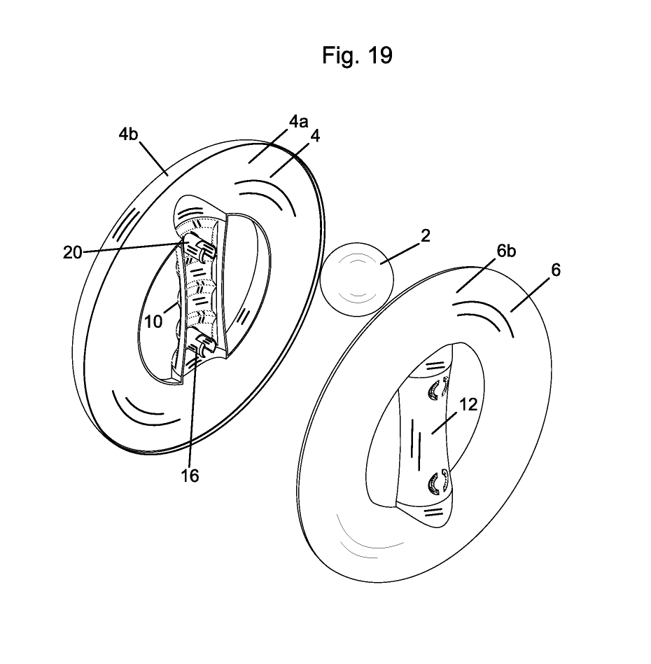

FIGS. 17, 18, and 19 show right side, front, and front perspective views of the apparatus 1 taken apart, with the structure 4, the further structure 6, and a ball 2 shown taken apart from each other.

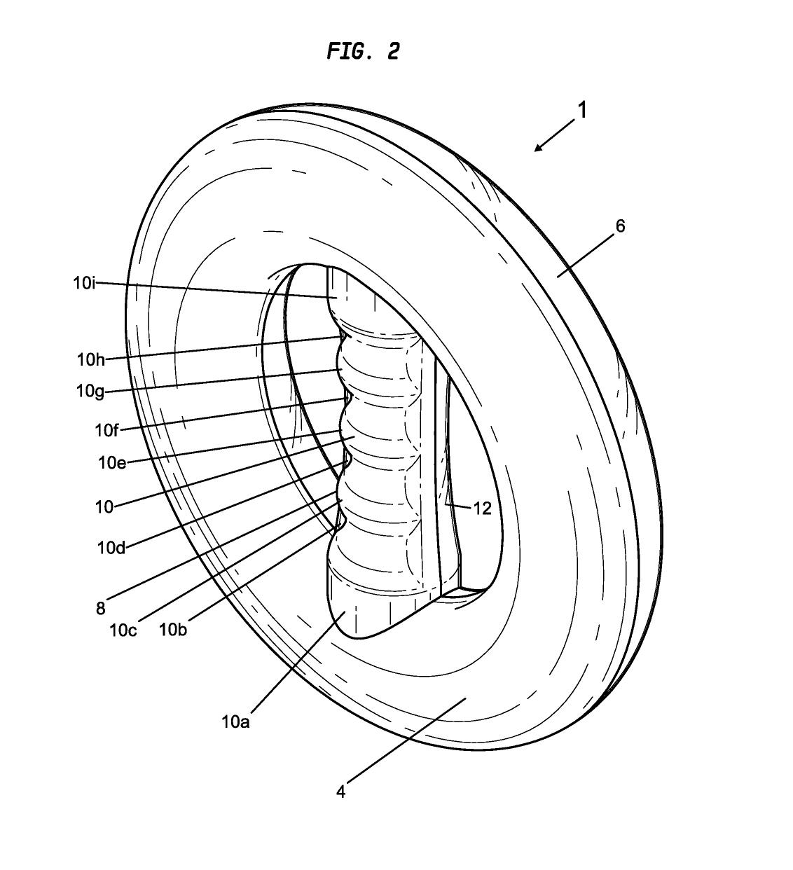

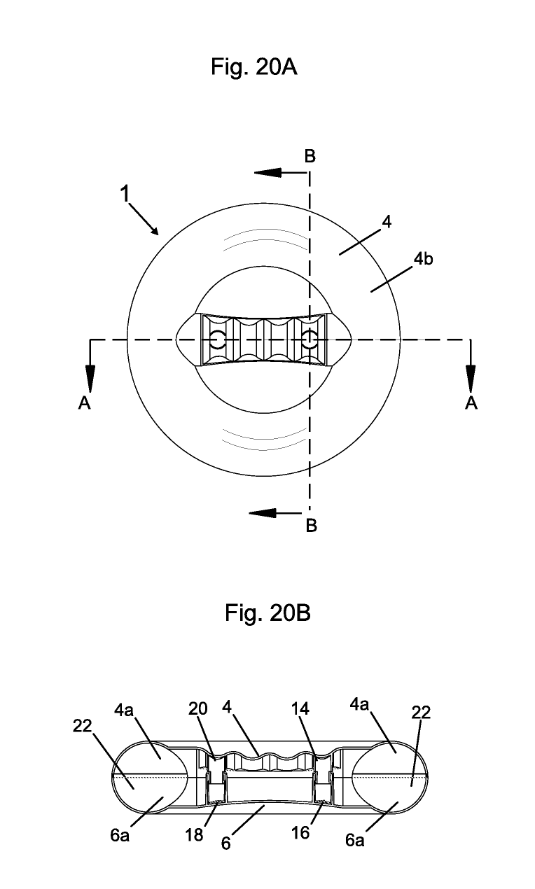

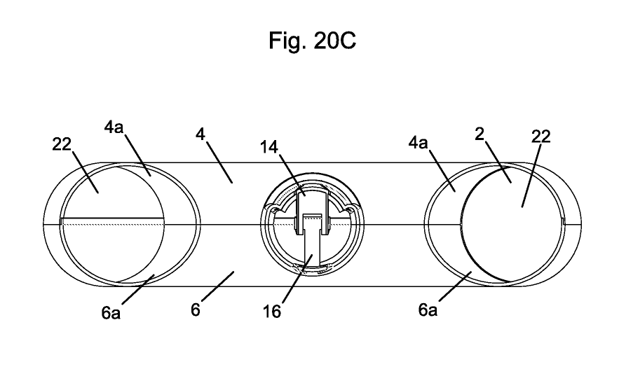

FIG. 20A shows a bottom view of the apparatus 1, with lines for sectional views of FIG. 20A and FIG. 20B. FIG. 20B shows a sectional view of the apparatus 1 along the line AA shown in FIG. 20A. FIG. 20C shows a sectional view of the apparatus 1 along the line BB shown in FIG. 20A Referring generally to FIGS. 1-20C, the apparatus 1 includes a ball 2, a structure 4, a structure 6, and a handle device 8. The handle device 8 includes a section 10 shown in FIG. 2 and a section 12 shown in FIG. 1. The section 10 of the handle device 8 includes portions 10a and 10i which are fixed to the structure 4, ridges 10c, 10e, and 10g, and recesses 10b, 10d, 10f, and 10h.

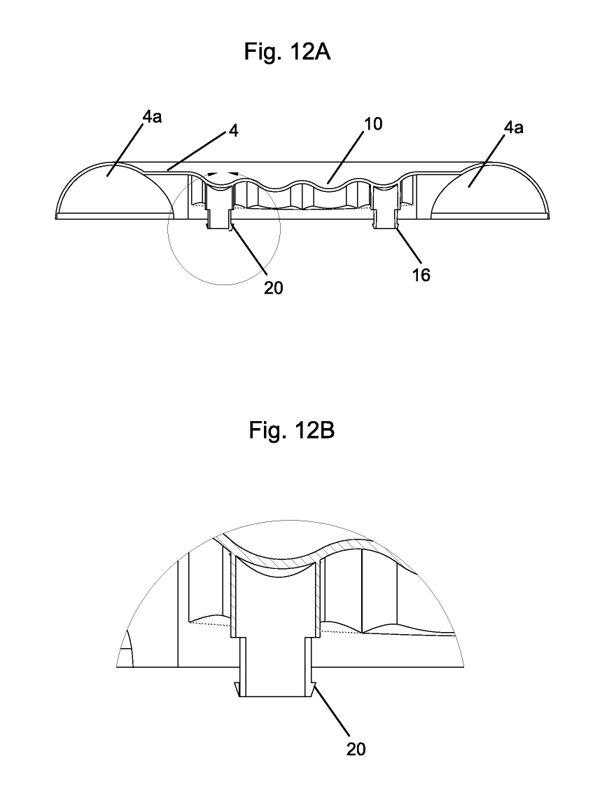

The apparatus 1 also includes keeper system portions 16 and 20 which are attached to the structure 4 as shown in FIG. 10, and keeper system portions 14 and 18 which are attached to the structure 6 as shown in FIG. 15. As shown in FIG. 20B, when the apparatus 1 is assembled the keeper portions 20 and 14 are inserted into the keeper portions 18 and 16, respectively, to connect and hold the structure 4 to the structure 6. As shown by FIGS. 12A and 12B, the portions 20 and 16 include a male aspect that has a distal flexible flange and a perpendicular slot. The keeper portions 20 and 16 engage the female portions 18 and 16, respectively, momentarily flexing the keeper portions 20 and 16 to accommodate the narrow channel of the portions 18 and 16 until the portions 20 and 14 reach the apex of the female openings of portions 18 and 16 where the flange clears the opening end. Through the resilient nature of the thermoplastic the portions 20 and 14 are held securely in place to the portions 18 and 16, respectively by the barbed flanges of the portions 20 and 14. This provides a significant benefit as no ultrasonic sealing or other complex manufacturing processes are necessary thereby reducing the cost of manufacturing while concurrently providing a secure and positive fit.

The structures 4 and 6 in the assembled form of FIGS. 1 and 2 form a hollow ring torus or hollow donut shape having an inner channel 22. The inner channel 22 is formed from channel portion 4a from structure 4 shown in FIG. 10 and channel portion 6a from structure 6 shown in FIG. 15. The ball 2 rolls and rotates freely in the inner channel 22 shown partially in sectional view of FIG. 20B, orbiting around the inner perimeter 5, shown in FIG. 1, of the combination of structures 4 and 6. Although it may appear from FIG. 1, that there are gaps in the inner channel 22 at the two connection ends of the handle device 8, in at least one embodiment, the inner channel 22 is completely smooth and continuous so that the ball 2 rolls smoothly three hundred and sixty degrees about the perimeter 5 in the inner channel 22.

In at least one embodiment, the handle device 8 is shut off from the typically circular inner channel 22, and the handle device 8 has a primary function of providing a central grip position which makes it easier to deliver different exercise movements and to make the ball 2 orbit the inner perimeter 5 while in the inner channel 22.

Although the structure 4 is shown as opaque, i.e. not transparent, in FIG. 1, and the structure 6 is shown as clear or transparent in FIG. 1, in alternative embodiments, the structure 4 may be transparent while the structure 6 may be opaque. In other embodiments, both the structures 4 and 6 may be transparent or both of the structures 4 and 6 may be opaque.

The structures 4 and 6 may be attached to form a round tube structure with a centrally located ergonomic handle device or structure 8. The handle device 8 may include ridges 10c, 10e, 10g, and recesses 10b, 10d, 10f, and 10h aggressive or frictional textures or materials so as to improve hand traction or friction. The ball 2 shown in FIG. 1, may be a cadence ball which may be made of dense rubber or rubber may be a rubber coated ball bearing.

In at least one embodiment, the structure 4 and/or the structure 6 may have a high impact color such as fluorescent green so as to give the user visual feedback and to encourage maintaining an exercise cadence as they rotate the ball in the inner channel 22 formed by the structure 4 and the structure 6.

Either of the structures 4 and 6 may be opaque and may be constructed of a thermoplastic that can be injection or rotationally molded. Either of the structures 4 and 6 may be made of an ABS (acrylonitrile butadiene styrene), polystyrene or other resilient material suitable for the application.

Either of the structures 4 and 6 may be a clear window and may be constructed of a K Resin® SBC, acrylic, polycarbonate or other suitable clear plastics. The ball or ball structure 2 may be manufactured of a butyl rubber, silicone or other elastomeric material. The ball 2 may also be constructed of a centralized metallic ball coated with a rubberized material. The ball 2 can also be manufactured of nylon, ABS and a wide range of suitable thermoplastics preferably with a smooth and not adhesive surface so as to facilitate ball movement inside the inner channel 22.

The design of the structures 4 and 6 is such so as to reduce manufacturing cost by eliminating slides in the apparatus 1. Generally, in the past when products are manufactured through an injection molding process, if the product is designed where there are holes or undercuts. slides need to come in after the injection mold is closed to create those detailed features. This significantly increases tooling cost while increasing production cycle times also having an adverse affect on unit price.

In contrast, in the present application, in at least one embodiment, the anticipated cycle time, meaning the time it takes to manufacture the apparatus 1 should be approximately thirty seconds. In at least one embodiment, the structures 4 and 6 have been configured to quickly snap together, using portions 24, 26, 28, 30, and 14, 16, 18, and 20, so that a person that is operating a molding press can install the ball 2 into the subassembly (which include structures 4 and 6) during that cycle time of thirty seconds. This significantly reduces unit cost and allows the product to be provided at a more affordable price to consumers.

In at least one embodiment, a textured finish on the base structure, such as one of structures 4 and 6, is designed to increase gripping traction, the textured finish on the handle assembly or handle device 8, is included on the clear structure (either structure 4 or 6) and base structure (either structure 4 or 6) so as to provide increased traction in the anterior and posterior section of the gripping surface, such as portion 10, of the handle device 8. The tapered structure of the gripping surface of the handle device 8 also increases human ergonomics by improving gripping traction.

In operation a person grips the handle device 8 with one hand, and moves the apparatus 1 causing the ball 2 to roll in the inner channel 22. The finger recesses 10b, 10d, 10f, and 10h, and/or the ridges 10c, 10e, and 10g increase tactile sensation and improve human ergonomics.

In at least one embodiment, when a user wears a fitness bracelet, exercises using the apparatus 1 and the apparatus 1 itself consistently activate an accelerometer of a fitness bracelet in order to catalog a more accurate exercise routine. Known exercise bracelets measure movement through a three axis or two axis accelerometer they also store this data until the user links it to his are her computer or portable connected device such as an iPhone or other many users of wearable devices will publish the results of their exercise routine on social networks such as Facebook or they will simply catalog their progression through provided application that is delivered with the wearable device and communicated through one or more wearable technologies or devices.

Most wearable devices communicate to smart phones through Bluetooth technologies such as Fitbit and Fuel band also have USB docking ports that allow individuals to upload the results to their desktop computers This provides a significant benefit to the user by providing them a more accurate measurement and catalog of fitness progression and fitness regimen compliance.

Although an exercise device of one or more embodiments of the present invention does not require any interplay with digital devices to be functional, its movements have been configured to correspond with the way in which an accelerometer measures arm movement. In one or more embodiments of the present invention, the apparatus 1 may be sold with access to online training videos that instruct users on how best to achieve optimal toning anaerobic fitness while leveraging movements that engage the accelerometer sensor located in a known wearable device, such as smart watch.

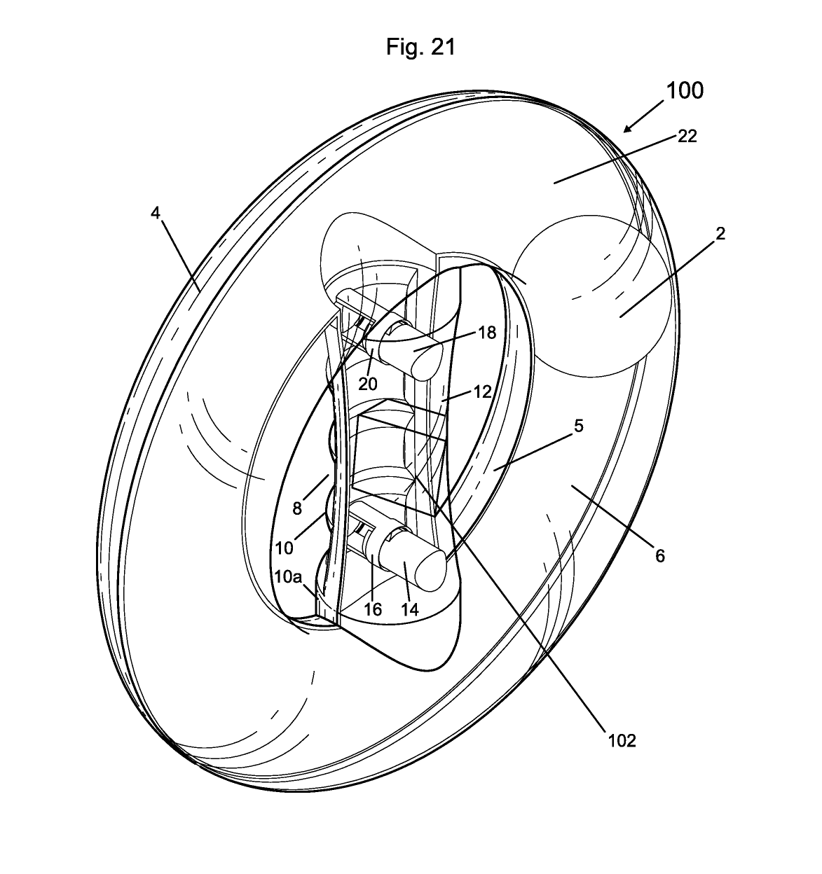

In at least one embodiment, a known accelerometer may be located inside the handle device 8. For example, as shown in a simplistic form, an apparatus 100 of FIG. 21 may be provided which is identical to the apparatus 1 of FIG. 1, with the exception of a container or box 102 embedded inside a cavity of the hollow handle device 8, wherein the box 102 includes various components including an accelerometer. The box 102 may include any component that a typical wearable device, such as a known smart watch would have. The box 102 may include a standard accelerometer with supporting circuitry to monitor and store movement in a computer or electronic memory, or to transmit this out similar to a smart watch. The box 102 may also include a USB connector so as to allow the apparatus 1 to charge an onboard rechargeable battery inside of the box 102 and upload movement data.

Alternatively a known accelerometer, and/or other electronics may be located in the ball 2. In an embodiment where an accelerometer is located in the ball 2 no wearable device would be required in order to monitor and measure body movement. This would provide a standalone integrated fitness device that includes the fitness apparatus 1 and the accelerometer or other suitable monitoring device.

The benefits of monitoring movement consistently are significant and include the ability for connected devices to aggregate data from a wearable wrist sensor or onboard data from an integrated configuration or design so as to measure the progression of fitness routines in order to determine the accurate level of exercise. In one or more embodiments, this aggregated data of a particular user is stored and compared. A wide range of commercially available bracelets are available that store movement data, in a computer or electronic memory, within the bracelet until they are linked, and/or transmitted out by some communication means. The communication means can include USB connectivity, Bluetooth and other conventional forms of wireless and wired connectivity. In the short term movement data may be stored in electronic or computer memory on the bracelet and ultimately long-range data may be stored in the cloud or on a some other computer device, such including a computer server.

Comparing data with data from other users provides a means to add game mechanics to the apparatus by way of movement competition. Studies have showed that game mechanics can increase utilization and performance of fitness devices as humans are inherently social and competitive. This data is so well known that large organizations are now deploying game mechanics in their health and wellness programs to drive compliance to fitness, weight loss and other health programs.

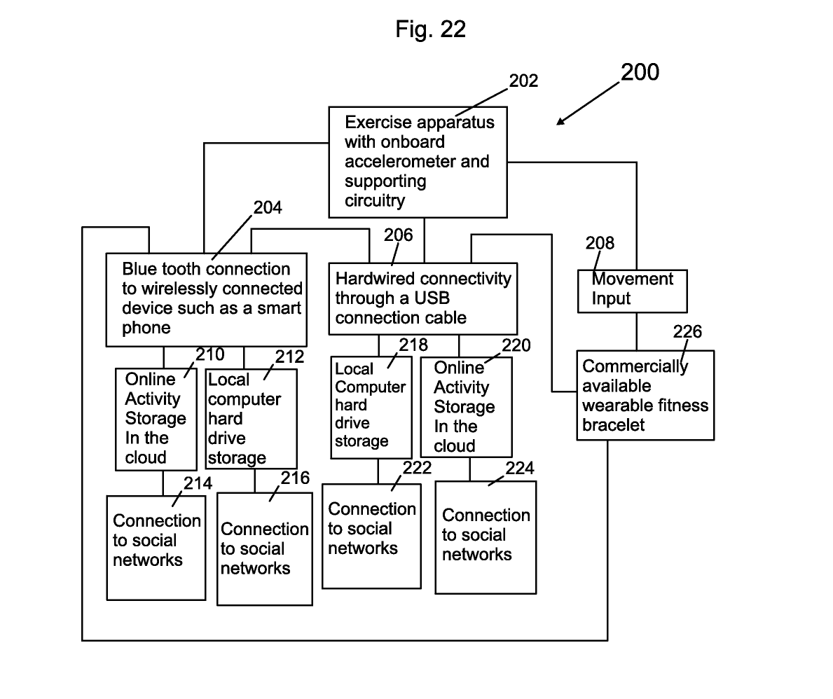

FIG. 22 shows a block diagram 200 of an apparatus, method, and/or system in accordance with another embodiment of the present invention. At step 202 an exercise apparatus, such as apparatus 1 in FIG. 1 or apparatus 100 in FIG. 21 may have or may be altered to include an onboard accelerometer and supporting circuitry such as would be found in a known smart watch or other known wearable device. The apparatus in step 202 may communicate by blue tooth technology and/or be wirelessly connected to a smart phone at step 204. The smart phone at step 204 may cause online activity storage in a computer memory or generally in the cloud of movement information of the apparatus 1 and/or apparatus 100 or of the apparatus of step 202, at step 210 The movement information may also be stored in computer memory of a local computer hard drive storage device at step 212. The movement information may be provided by connection to social networks at either steps 214 or 216.

Movement information from step 202 and/or from apparatus 1 or 100, may be communicated through hardwired connectivity through a USB connection cable which may for example be connected to box 102 in FIG. 21. Movement information may be stored in a local computer hard drive at step 218 or on the cloud at step 220. The movement information may be provided by connection to social networks at either steps 222 or 224.

Movement information may be provided from apparatus 1 or 100 or from step 202 to a known commercially available wearable fitness bracelet through steps 208 and 226. The fitness bracelet may then provide data by any known means such as hardwired or wireless to steps or devices 210, 212, 218, and 220.

The dangers of a sedentary lifestyle are well-established in the medical literature. One or more embodiments of the present invention, such as including apparatus 1 and apparatus 100 and/or the system, apparatus, and method of FIG. 22, provides the ability to simultaneously use eccentric and concentric muscle strengthening through targeted exercise. This in turn, provides a fantastic base strength for activities of daily living which can improve the resistance to injury.

Eccentric muscle strengthening has also been shown to help a tendon remodel in cases of tendinopathy/tendinitis. Eccentric strengthening means strengthening while a muscle is elongating. For instance, the negative portion of a biceps curl. The biceps is lengthening while the resistance is present. The apparatus 1 and/or apparatus 100, and/or system, method, and apparatus 200 also prevent potential injuries to the joints when compared to lifting heavy weights.

Aerobic benefits are achieved when the apparatus 1 and/or 100 is used in conjunction with a series of dance like exercise programs similar to those in dance program such as Zumba (trademarked) and Jazzercise (trademarked) all those specific workouts that have been created that when done with apparatus 1 and/or 100 encourage a rapid rotation of the ball 2 during dance and exercise routines.

Examples of dance and fitness routines which can be done with the apparatus 1 and/or 100 in accordance with an embodiment of the present invention include:

(a) Front rotation exercise—by grasping the central handle or handle device 8 and positioning the apparatus 1 or apparatus 100 at a ninety degree angle from a person's arm, the subject can rotate the ball 2 in small concentric circles gradually migrating to large sweeping circles. This movement builds the muscles around the shoulder and pectoral muscles while concurrently delivering an aerobic exercise routine.

(b) Helicopter rotational exercise—while grasping the central handle or handle device 8 of the apparatus 1 or apparatus 100, the individual rotates the apparatus 1 and/or 100 above their head similar to that of a helicopter rotor; and this provides lateral fitness, shoulder and core while concurrently delivering an aerobic exercise routine.

(c) Arms out rotational exercise—while grasping the central handle or handle device 8 while both hands are positioned to the side and the subject rotates in small and large circles to build core, lateral and shoulder muscles while concurrently delivering an aerobic exercise routine.

(d) One or more embodiments of the present invention can also be used for slow-moving routines that focus on pre-workout stretching similar to Pilates and yoga. The slow rotation of the ball 2 helps the subject focus on the stretching movement and provides tactile feedback through the sensation of the weight of the ball 2 weight. Dozens of stretching exercises can be achieved using the subject invention as the ball 2 provides visual and tactile feedback to the subject to assist them in proper body positioning.

(e) Hammer and thrusting exercises—while holding the device in the ring area perpendicular to the arm, the subject can swing the device in a range of exercises to provide bicep and tricep strength training while concurrently enjoying the benefits of an aerobic exercise routine.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. An apparatus comprising
a ring shaped structure having a ring shaped inner channel; and
a ball which is located inside the ring shaped structure and which rolls in the ring shaped channel.

2. The apparatus of claim 1 wherein
the ring shaped structure is comprised of a first ring shaped structure and a second ring shaped structure which are attached together to form the ring shaped structure.

3. The apparatus of claim 1 wherein
the ring shaped structure has an inner diameter;
and the apparatus further comprising a handle which spans the inner diameter of the ring shaped structure.

4. The apparatus of claim 2 wherein
the ring shaped structure has an inner diameter;
and the apparatus further comprising a handle which spans the inner diameter of the ring shaped structure;
a first attachment device; and
a second attachment device; and
wherein the first attachment device connects to the second attachment device inside of the handle to attach the first ring shaped structure to the second ring shaped structure.

5. The apparatus of claim 4 wherein
the first attachment device and the second attachment device are substantially perpendicular to the handle.

6. The apparatus of claim 1 further comprising
a device for measuring movement of the ball.

7. The apparatus of claim 6 wherein
the device for measuring movement of the ball includes an accelerometer.

8. The apparatus of claim 6 wherein
the device for measuring movement of the ball includes electronic memory for storing data related to the movement of the ball.

9. The apparatus of claim 6 wherein
the device for measuring movement of the ball includes a transmitter for transmitting out data related to the movement of the ball.

10. The apparatus of claim 6 wherein
the device for measuring movement of the ball includes a port for interfacing with a computer device for downloading data related to movement of the ball.

11. The apparatus of claim 1 further comprising
a wearable electronic device configured to be attached to a human hand, and which monitors movement of the human hand, when the wearable electronic device is attached to the human hand.

12. A method comprising
gripping a ring shaped structure with a human hand;
moving the human hand while gripping the ring shaped structure; and
wherein the ring shaped structure has a ring shaped inner channel, and a ball is located in the ring shaped inner channel; and
wherein the ball rolls in the ring shaped inner channel when the human hand is moved while gripping the ring shaped structure.

13. The method of claim 12 wherein
the ring shaped structure is comprised of a first ring shaped structure and a second ring shaped structure which are attached together to form the ring shaped structure.

14. The method of claim 12 wherein
the ring shaped structure has an inner diameter;
a handle spans the inner diameter of the ring shaped structure;
and wherein the ring shaped structure is gripped by the human hand gripping the handle.

15. The method of claim 13 wherein
the ring shaped structure has an inner diameter;
a handle which spans the inner diameter of the ring shaped structure;
wherein the ring shaped structure is gripped by the human hand gripping the handle;
and wherein a first attachment device connects to a second attachment device inside of the handle to attach the first ring shaped structure to the second ring shaped structure.

16. The method of claim 15 wherein
the first attachment device and the second attachment device are substantially perpendicular to the handle.

17. The method of claim 12 further comprising
measuring movement of the ball.

18. The method of claim 17 further comprising
storing data concerning the movement of the ball in electronic memory.

19. The method of claim 17 further comprising
sending out data concerning movement of the ball.

20. The method of claim 12 further comprising
attaching a wearable electronic device to the human hand, and monitoring movement of the human hand, when the wearable electronic device is attached to the human hand and while the human is gripping the ring shaped structure.
